(19) United States
(12) Patent Application Publication
(10) Pub. No.: US 2006/0265005 A1
(43) Pub. Date: Nov. 23, 2006

(54) Kit and a method for closing a wound

(76) Inventor: Stephen A. Beese, Floyd, VA (US)

Correspondence Address: HAMMER & HANF, PC, 3125 SPRINGBANK LANE, SUITE G, CHARLOTTE, NC, 28226, US

(21) Appl. No.: 11/133,980

(22) Filed: May 20, 2005

(51) Int. Cl. A61B 17/08

(52) U.S. Cl. 606/215

(57) ABSTRACT

The instant invention is a kit and a method for closing a wound. The kit for closing a wound includes at least one transparent support layer, and a flowable adhesive. The support layer includes a pressure-sensitive adhesive coating on one side thereof. The method for closing a wound includes the following steps: (1) providing a kit for closing a wound which includes at least one transparent support layer that has a pressure-sensitive adhesive coating on one side thereof, and a flowable adhesive; (2) aligning skin edges of the wound thereby juxtaposing the opposite margins of the wound next to each other; (3) affixing the transparent support layer on the juxtaposed margins of the wound thereby securing the opposite margins of the wound in the juxtaposed position; (4) applying the flowable adhesive to the exposed portions of the support layer and the wound; (5) allowing the adhesive to solidify; and (6) thereby closing the wound.

### FIELD OF INVENTION

The instant application relates to a kit and a method for closing a wound.

### BACKGROUND OF THE INVENTION

The use of sutures, adhesive skin tapes, surgical staples, and topical adhesives to close a wound or an incision is generally well known. However, there are many limitations associated with each of these techniques. Sutures, for example, cause additional skin trauma during placement, leave wound edges exposed thereby allowing bacterial access and possibility of infection, and create unattractive wound closure marks. Adhesive skin tapes also leave wound edges exposed thereby allowing bacterial access and possibility of infection. Surgical staples, for example, cause pain during application, result in scarring, fail to allow repositioning of the wound without their removal, and require the patient to return to the healthcare provider for their removal. Topical adhesives, on the other hand, are not user friendly because they require a substantial amount of skills and efforts to maintain the wound edges in their proper anatomic positions during the application thereof. Furthermore, topical adhesive require the healthcare provider to reposition the wound surface as close to the horizontal surface as possible. Additionally, the current techniques fail to properly maintain a tight juxtaposition and a proper anatomic alignment of the wound edges during the application of topical adhesives to the wound.

U.S. Pat. No. 5,047,047 discloses a wound closing device including a body having a first arm and a second arm, and at least one skin engaging member on each arm to penetrate the skin surrounding the wound.

U.S. Pat. No. 5,259,835 discloses a wound closure device employing a porous bonding membrane, which receives a flowable adhesive.

U.S. Pat. No. 5,263,970 discloses a surgical dressing for closing a wound, which includes a pair of locating members to be placed on opposed sides of a wound. A manipulating means interconnects the locating members, and draws the locating members toward each other to close the wound.

U.S. Pat. No. 6,270,530 discloses a prosthetic repair fabric including a sheet of infiltratable fabric and a second sheet united thereto. The second sheet is fused to an adhesion resistant barrier forming a laminate composite prosthesis.

U.S. Pat. No. 6,329,564 discloses a bandage for closing a wound or incision. The bandage includes a first flat flexible component having adhesive on a lower surface and a plurality of first elongated connectors extending from one edge thereof in a first direction. The bandage further comprises a second flat flexible component having adhesive on a lower surface and one or more second elongated connectors extending from one edge thereof in a second direction generally opposite to the first direction. Pulling elements are joined to the first and second elongated connectors. Means are provided for attaching the first elongated connectors to the second flat flexible component and the second elongated connectors to the first flat flexible component.

U.S. Pat. No. 6,652,559 discloses a wound closure system. The wound closure system includes an elongated flexible backing strip. The backing strip includes a first portion disposed between the ends and adapted to overlie the facing edges of the wound. A second and third portions are disposed on either side of the first portion, and each provided a predetermined number of spaced-apart apertures extending through the backing strip from one surface to the other. A first pressure-sensitive adhesive is coated on at least part of the first surface of the backing strip including the second and third portions thereof. The wound closure system further includes a flowable, moisture-curable surgical adhesive for application into the apertures to strengthen the adhesion of the second and third portions of the backing strip to the patient.

Despite the research efforts in developing different wound closure systems, there is still a need for a wound closure system that facilitates proper wound closure in a relatively short period of time, which also minimizes any scarring. Furthermore, there is still a need for a method for closing a wound, which facilitates proper wound closure in a relatively short period of time, which also minimizes any scarring.

### SUMMARY OF THE INVENTION

The instant invention is a kit and a method for closing a wound. The kit for closing a wound includes at least one transparent support layer, and a flowable adhesive. The support layer includes a pressure-sensitive adhesive coating on one side thereof. The method for closing a wound includes the following steps: (1) providing a kit for closing a wound which includes at least one transparent support layer that has a pressure-sensitive adhesive coating on one side thereof, and a flowable adhesive; (2) aligning skin edges of the wound thereby juxtaposing the opposite margins of the wound next to each other; (3) affixing the transparent support layer on the juxtaposed margins of the wound thereby securing the opposite margins of the wound in the juxtaposed position; (4) applying the flowable adhesive to the exposed portions of the support layer and the wound; (5) allowing the adhesive to solidify; and (6) thereby closing the wound.

### BRIEF DESCRIPTION OF THE DRAWINGS

For the purpose of illustrating the invention, there is shown in the drawings a form that is presently preferred; it being understood, however, that this invention is not limited to the precise arrangements and instrumentalities shown.

FIG. 1 is a schematic illustration of a kit for closing a wound according to instant invention;

FIG. 2 is an alternative schematic illustration of a kit for closing a wound according to instant invention;

FIG. 3 is a cross-sectional view of a coated transparent support layer;

FIGS. 4A-B are cross-sectional views of alternative coated transparent support layers;

FIGS. 5A-E are schematic illustration of different embodiments of transparent support layer;

FIG. 6 is a schematic illustration of how the adhesive release cover of FIG. 3B may be removed;

FIG. 7 is a first schematic illustration of how to close a wound according to the instant invention;

FIG. 8 is a second schematic illustration of how to close a wound according to the instant invention; and

FIG. 9 is a third schematic illustration of how to close a wound according to the instant invention.

### DETAILED DESCRIPTION OF THE INVENTION

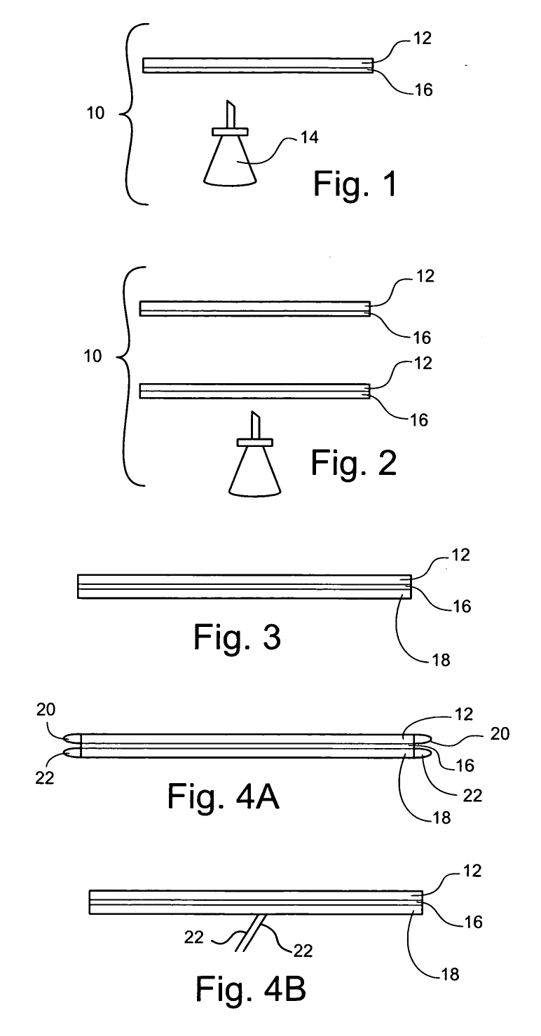

Referring to the drawings wherein like numerals indicate like elements, there is shown, in FIG. 1, a preferred embodiment of kit 10 for closing a wound. Kit 10 for closing a wound includes at least one transparent support layer 12, and a flowable adhesive 14. The transparent support layer 12 may further include a pressure-sensitive adhesive coating 16, and an adhesive release cover 18, as shown in FIG. 3.

Transparent, as used herein, refers to any material of such fine or open texture that objects may be easily seen on the other side.

The instant invention, for convenience, has been described in terms of a kit for closing a wound; however, the instant invention is not so limited, and it may, for example, be employed for closing a surgical incision. Furthermore, the instant invention may be employed to close a wound or a surgical incision in a human being or an animal, e.g. a horse.

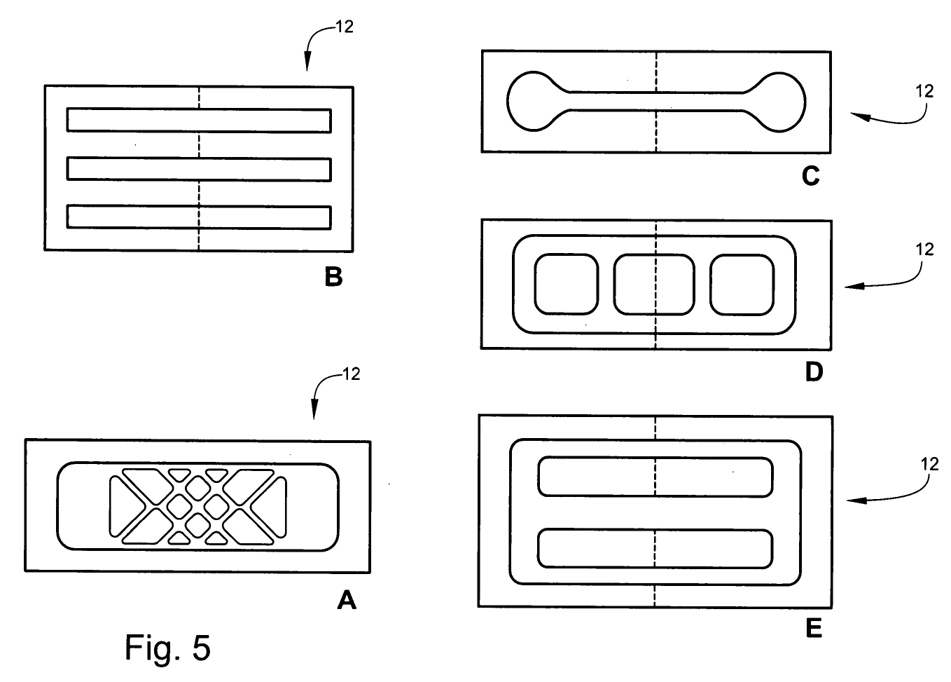

Referring to FIG. 1, Kit 10 includes at least one transparent support layer 12. Referring to FIG. 2, kit 10 may include a plurality of support layers 12. For example, kit 10, shown in FIG. 2, may have two support layers 12. Kit 10 may include a plurality of identical support layers 12, e.g. same design and same material, unidentical support layers 12, e.g. different designs or different materials, or combinations thereof. Referring to FIGS. 3-5, the transparent support layer 12 may have any size. For example, the transparent support layer 12 may be adapted to cover an entire wound, e.g. a patch or a tape, or it may be adapted to only cover a small portion of a wound, e.g. a strip. The transparent support layer may have any shape adapted to cover a wound. For example, the transparent support layer 12 may have a shape selected from the group consisting of a square, a rectangle, a circle, a triangle, combinations thereof, and the like. The exemplary listed shapes are not regarded as limiting. The transparent support layer 12 may be any material. For example, transparent support layer 12 may be a material selected from the group consisting of a film, a woven, a nonwoven, a mesh foil, and combinations thereof. A film, for example, may be a polymeric film such as a polyethylene film. A woven material is a fabric composed of two sets of yarns interlaced with each other to form the fabric. A non-woven material is the assembly of textile fibers held together by mechanical interlocking in a random web or mat, for example by fusing thermoplastic fibers to each other. A mesh foil may be any metallic material. The exemplary listed materials for transparent support layer 12 are not regarded as limiting. Transparent support layer 12 may be permeable, impermeable, or combinations thereof. For example, transparent support layer 12 may be porous or microporous. In the alternative, it may include at least one aperture, and preferably, it may include a plurality of apertures. Transparent support layer 12 may be adapted to facilitate the application thereof. For example, transparent support layer 12 may further include a flap portion 20; in the alternative, transparent support layer 12 may further include a plurality of flap portions 20, as shown in FIG. 4A. Flap portions 20 may be any material; for example, flap portion 20 may be a material selected from the group consisting of a film, a woven, a nonwoven, a mesh foil, and combinations thereof. Flap portion 20 may be an integral component of transparent support layer 12; or in the alternative, flap portion 20 may be a separate individual component secured to the transparent support layer 12 via conventional methods, e.g. adhesive, heat bonding, stitching and the like. Flap portion 20 is, preferably, adhesive coating free.

Referring to FIG. 3, transparent support layer 12 further includes an adhesive coating 16 on one surface. Adhesive coating 16 may be any biocompatible adhesive. Adhesive coating 16 may be any biocompatible adhesive coating material suitable for forming a bond between skin and the transparent support layer 12. Skin, as used herein, refers to both human skin, and animal skin. Adhesive coating 16 may, for example, be a pressure-sensitive adhesive.

Referring to FIG. 3, transparent support layer 12 may further include an adhesive release cover 18. Adhesive release cover 18 may be any conventional release cover; for example, adhesive release cover 18 may be a film, e.g. polymeric film. Adhesive release cover 18 may be adapted to facilitate the application of the transparent support layer 12 with minimum amount of complications. For example, adhesive release cover 18 may further include a flap portion 22; in the alternative, adhesive release cover 18 may further include a plurality of flap portions 22, as shown in FIG. 4. Flap portions 22 may be any material; for example, flap portion 22 may be a material selected from the group consisting of a film, a woven, a nonwoven, a mesh foil, and combinations thereof. Flap portion 22 may be an integral component of adhesive release cover 18; or in the alternative, flap portion 22 may be a separate individual component secured to the adhesive release cover 18 via conventional methods, e.g. adhesive, heat bonding, stitching and the like. Flap portion 22 is, preferably, adhesive coating free. Flap portion 22 may be placed on any portion of adhesive release cover 18, e.g. end point of adhesive release cover 18, midpoint of adhesive release cover 18, and any point therebetween the end points.

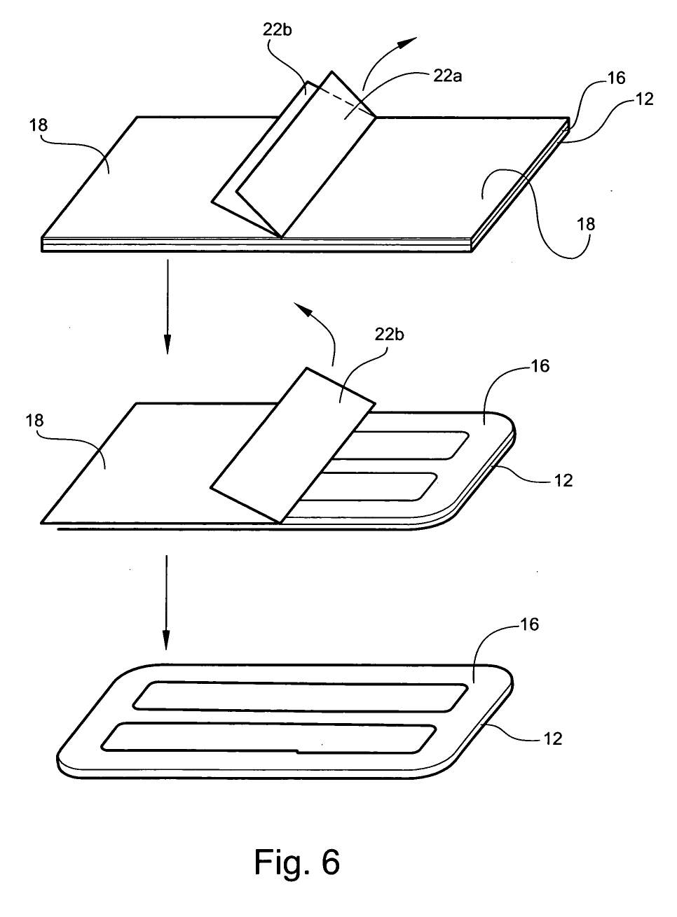

Referring to FIG. 6, adhesive release cover 18 may, for example, be removed in sections. Initially, a first portion of adhesive release cover 18 may be removed via a first flap portion 22a, and then the second portion of the adhesive release paper may be removed via a second flap portion 22b. In the alternative, the entire adhesive release cover 18 may be removed at once.

Flowable adhesive 14 may be any biocompatible adhesive suitable for wound application. Flowable, as used herein, refers to the tendency of the adhesive placed on the higher end of an incline to move to the lower end of the incline. Flowable adhesive 14 may be a liquid, a semi-solid, a gelatinous paste, a suspension, or combinations thereof. Flowable adhesive may have any viscosity; for example, flowable adhesive may have a low viscosity, a medium viscosity, or a high viscosity. Flowable adhesive may be applied via any method; for example, pouring, spraying, sprinkling, brushing, and the like. Flowable adhesive 14 may be a monomer capable of polymerizing into a tough, flexible film that is capable of adhering to both skin and the transparent support layer 12. Flowable adhesive 14 may, for example, be a biocompatible cyanoacrylate, a biocompatible silicone based adhesive, or the like. Flowable adhesive 14 may be a flowable adhesive adapted to be applied to human skin or animal skin. Flowable adhesives 14 adapted to be applied to human skin are commercially available under the names DERMABOND® (2-octyl-cynaoacrylate) from Ethicon, Inc. of West Summerville, N.J., INDERMIL® (n-Butyl-2-cynoacrylate) from Tyco Healthcare of Mansfield, Mass., HISTOACRYL BLUE® from B. Baurn of Melsungen, Germany, LIQUIBAND® (2-octyl-cynoacrylate) from Medlogic Global Limited of Plymouth, United Kingdom, EPIGLU® (ethyl-2-cynoacrylate) from Meyer-Haake Medical Innovations of Wehrhelm, Germany, DOW CORNING® 7-9800 Soft Skin Adhesive Kit (A&B) from Dow Corning Corp. of Midland, Mich., and BIOGLUE® Surgical Adhesive from Cryolife of Kennesaw, Ga. Flowable adhesives 14 adapted to be applied to animal skin are commercially available under the names VETBOND TISSUE ADHESIVE® from 3M Healthcare, of St. Paul, Minn., and NEXBAND® from Closure Medical Corp of Raleigh, N.C.

Kit 10 may further include an indicator factor (not shown) to prompt the completion of the polymerization reaction of the flowable adhesive 14. Indicator factor may be any agent adapted to prompt the completion of the polymerization reaction of the flowable adhesive 14. For example, indicator factor may be a color indicator that changes, for example, color or intensity to prompt the completion of the polymerization reaction of the flowable adhesive 14. Indicator factor may be any biocompatible indicator factor. Such indicator factors are generally well known in the art. Indicator factor may be an integral component of the flowable adhesive 14, or it may be a separate individual component to be added to adhesive 14 immediately prior to application thereof, to be applied to the support layer 12 immediately before the application of flowable adhesive 14 thereto, or to be applied to the flowable adhesive 14 immediately after the application thereof. In the alternative, the indicator factor may be a coating on the surface of the transparent support layer 12; preferably, on the surface that does not carry the adhesive coating 16, or it may be an integral component of the transparent support layer 12.

Kit 10 for closing a wound may further include a catalyst (not shown) to facilitate the acceleration of polymerization reaction of the flowable adhesive 14. Such catalysts are generally well known in the art. Exemplary catalysts include, but are not limited to, Polyethylene glycol (“PEG”) derivatives such as Poly(oxy-1,2-ethanediyl), and a-methyl-w-methoxy-(Poly(ethylene glycol). Catalyst may be an integral component of the flowable adhesive 14, or it may be a separate individual component to be added to adhesive 14 immediately prior to application thereof, to be applied to the support layer 12 immediately before the application of flowable adhesive 14 thereto, or to be applied to the flowable adhesive 14 immediately after the application thereof. In the alternative, the catalyst may be a coating on the surface of the transparent support layer 12; preferably, on the surface that does not carry the adhesive coating 16. The catalyst may also be combined with the indicator factor.

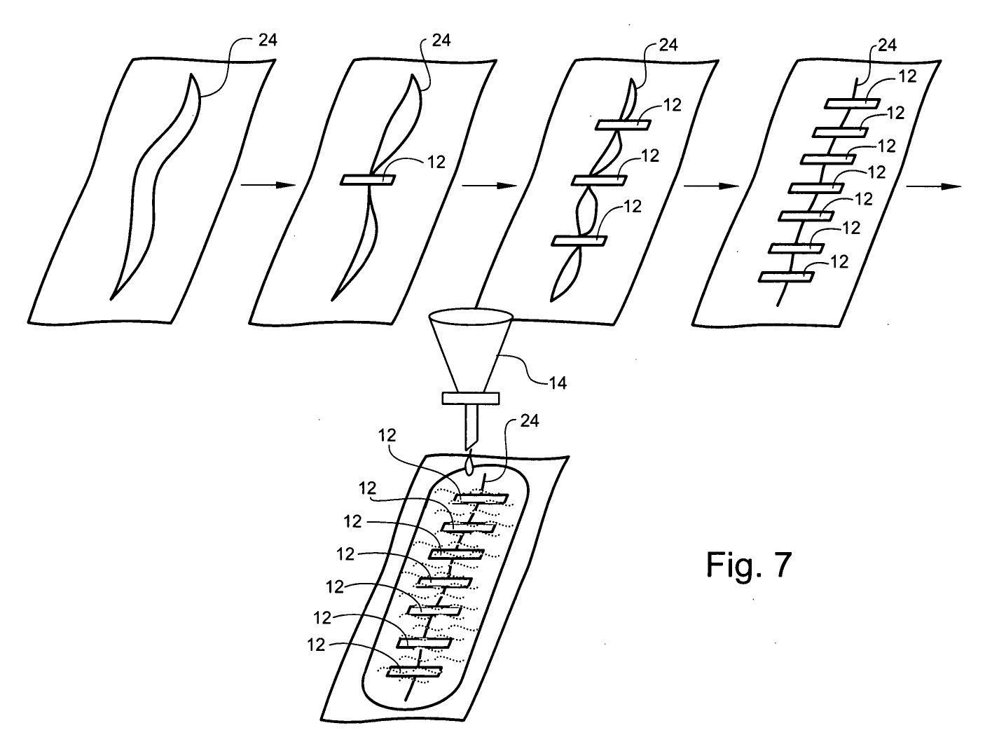

In operation, referring to FIG. 7, a healthcare provider properly aligns the edges of the wound of a patient thereby juxtaposing the opposite margins of the wound next to each other in their correct anatomic position; subsequently, the healthcare provider secures the juxtaposed opposite margins of the wound of the patient by affixing the transparent support layer 12, e.g. a strip shape support layer, on the opposite margins of the wound. The healthcare provider continues to repeat the aligning of the edges of the wound of the patient thereby juxtaposing the opposite margins of the wound next to each other, and then securing them in place via transparent support layer 12 until the entire length of the opposite margins of the wound is secured in a proper juxtaposed position. The transparent support layer 12 allows the healthcare provider to examine the position of the opposite margins of the wound thereby assuring that the opposite margins of the wound are properly juxtaposed next to each other. If the healthcare provider determines that the opposite margins of the wound are not properly juxtaposed next to each other, then the healthcare provider may remove the entire transparent support layer 12 or a portion thereof, and realign the opposite margins of the wound thereby juxtaposing them next to each other; subsequently, securing them in their juxtaposed position via transparent support layer 12. Once the healthcare provider determines that the opposite margins of the wound are secured in their proper juxtaposed position, the healthcare provider applies the flowable adhesive 14 to the exposed portions of the transparent support layer 12 and the wound. The flowable adhesive 14 is allowed to polymerize and solidify into a tough, flexible film adhering to both the skin and the transparent support layer 12. If an indicator factor, e.g. a color indicator, is present, then a change in color prompts the completion of the polymerization reaction, i.e. the flowable adhesive is solidified. The polymerization reaction of the flowable adhesive 14 may be accelerated via a catalyst, as discussed hereinabove. The transparent support layer 12 acts as a frame providing strength to the flowable adhesive 14, and it allows the healthcare provider to examine the position of the margins of the wound thereby assuring that the opposite margins of the wounds are properly juxtaposed next to each other.

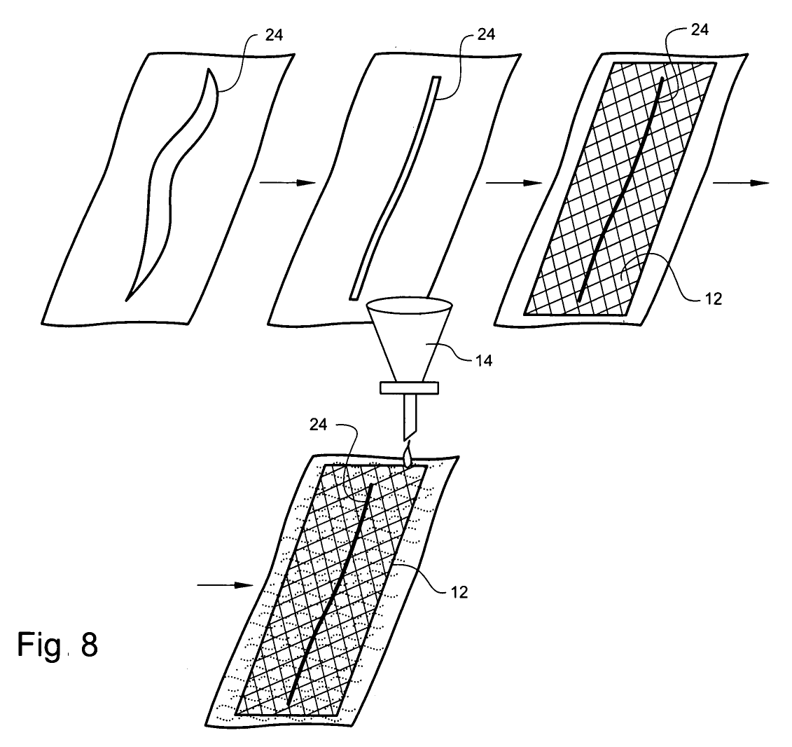

In alternative operation, referring to FIG. 8, a healthcare provider aligns the edges of the wound of a patient thereby juxtaposing the opposite margins of the wound next to each other; subsequently, the healthcare provider secures the juxtaposed opposite margins of the wound of the patient by affixing the transparent support layer 12, e.g. a rectangle shape support layer, on the opposite margins of the wound. The healthcare provider continues to repeat the aligning of the edges of the wound of the patient thereby juxtaposing the opposite margins of the wound next to each other, subsequently, securing them in place via transparent support layer 12 until the entire length of the opposite margins of the wound is secured in a proper juxtaposed position. The transparent support layer 12 allows the healthcare provider to examine the position of the opposite margins of the wound thereby assuring that the opposite margins of the wound are properly juxtaposed next to each other. If the healthcare provider determines that the opposite margins of the wound are not properly juxtaposed next to each other, then the healthcare provider may remove the entire transparent support layer 12 or a portion thereof, and realign the opposite margins of the wound thereby juxtaposing them next to each other, and subsequently, securing them in their proper juxtaposed position via transparent support layer 12. Once the healthcare provider determines that the opposite margins of the wound are secured in their proper juxtaposed position, the healthcare provider applies the flowable adhesive 14 to the exposed portions of the transparent support layer 12 and the wound. The flowable adhesive 14 is allowed to polymerize and solidify into a tough, flexible film adhering to both the skin and the transparent support layer 12. If an indicator factor, e.g. a color indicator, is present, then a change in color prompts the completion of the polymerization reaction, i.e. the flowable adhesive is solidified. The polymerization reaction of the flowable adhesive 14 may be accelerated via a catalyst, as discussed hereinabove. The transparent support layer 12 acts as a frame providing strength to the flowable adhesive 14, and it allows the healthcare provider to examine the position of the margins of the wound thereby assuring that the opposite margins of the wounds are properly juxtaposed next to each other.

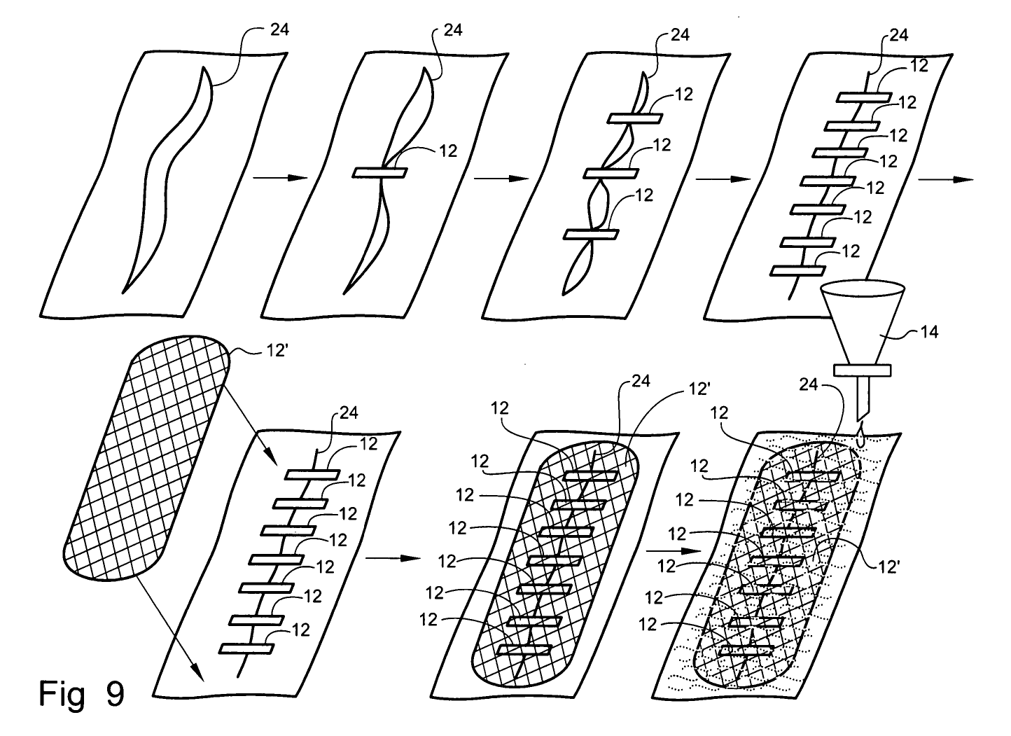

In another alternative operation, referring to FIG. 9, a healthcare provider properly aligns the edges of the wound of a patient thereby juxtaposing the opposite margins of the wound next to each other in their correct anatomic position; subsequently, the healthcare provider secures the juxtaposed opposite margins of the wound of the patient by affixing the transparent support layer 12, e.g. a strip shape support layer, on the opposite margins of the wound. The healthcare provider continues to repeat the aligning of the edges of the wound of the patient thereby juxtaposing the opposite margins of the wound next to each other, and then securing them in place via transparent support layer 12 until the entire length of the opposite margins of the wound is secured in a proper juxtaposed position. The transparent support layer 12 allows the healthcare provider to examine the position of the opposite margins of the wound thereby assuring that the opposite margins of the wound are properly juxtaposed next to each other. If the healthcare provider determines that the opposite margins of the wound are not properly juxtaposed next to each other, then the healthcare provider may remove the entire transparent support layer 12 or a portion thereof, and realign the opposite margins of the wound thereby juxtaposing them next to each other; subsequently, securing them in their juxtaposed position via transparent support layer 12. Once the healthcare provider determines that the opposite margins of the wound are secured in their proper juxtaposed position, the healthcare provider may place a second transparent support layer 12 upon secured wound, and subsequently, the healthcare provider applies the flowable adhesive 14 to the exposed portions of both transparent support layers 12 and the wound. The flowable adhesive 14 is allowed to polymerize and solidify into a tough, flexible film adhering to both the skin and both of the transparent support layers 12. If an indicator factor, e.g. a color indicator, is present, then a change in color prompts the completion of the polymerization reaction, i.e. the flowable adhesive is solidified. The polymerization reaction of the flowable adhesive 14 may be accelerated via a catalyst, as discussed hereinabove. Both of the transparent support layers 12 act as a frame providing strength to the flowable adhesive 14, and they allow the healthcare provider to examine the position of the margins of the wound thereby assuring that the opposite margins of the wounds are properly juxtaposed next to each other.

The present invention may be embodied in other forms without departing from the spirit and the essential attributes thereof, and, accordingly, reference should be made to the appended claims, rather than to the foregoing specification, as indicated the scope of the invention.

We claim:

1. A kit for closing a wound comprising:
at least one transparent support layer, said support layer having a pressure-sensitive adhesive coating on one side; and
a flowable adhesive.

2. The kit for closing a wound according to claim 1, wherein said transparent support layer being permeable, impermeable, or combinations thereof.

3. The kit for closing a wound according to claim 1, wherein said transparent support layer being a material selected from the group consisting of a film, a woven, a nonwoven, a mesh foil, and combinations thereof.

4. The kit for closing a wound according to claim 1, wherein said flowable adhesive being a biocompatible cyanoacrylate adhesive, a biocompatible silicone based adhesive, or the like.

5. The kit for closing a wound according to claim 1, wherein said kit further including an indicator factor.

6. The kit for closing a wound according to claim 5, wherein said indicator factor being a color factor.

7. The kit for closing a wound according to claim 1, wherein said kit further including a catalyst.

8. The kit for closing a wound according to claim 7, wherein said catalyst being a Polyethylene glycol derivative.

9. A method for closing a wound comprising the steps of:
providing a kit for closing a wound comprising:
at least one transparent support layer, said transparent support layer having a pressure-sensitive adhesive coating on one side; and
a flowable adhesive;
aligning skin edges of said wound thereby properly juxtaposing opposite margins of said wound next to each other;
affixing said transparent support layer on said juxtaposed margins of said wound thereby securing said opposite margins of said wound in said proper juxtaposed position;
applying said flowable adhesive to exposed portions of said support layer and said wound;
allowing said adhesive to solidify; and
thereby closing said wound.

10. The method for closing a wound according to claim 9, wherein said transparent support layer being permeable, impermeable, or combinations thereof.

11. The method for closing a wound according to claim 9, wherein said transparent support layer being a material selected from the group consisting of a film, a woven, a nonwoven, a mesh foil, and combinations thereof.

12. The method for closing a wound according to claim 9, wherein said flowable adhesive being a biocompatible cyanoacrylate adhesive, a biocompatible silicone based adhesive, or the like.

13. The method for closing a wound according to claim 9, wherein said kit further including an indicator factor.

14. The method for closing a wound according to claim 13, wherein said indicator factor being a color factor.

15. The method for closing a wound according to claim 9, wherein said kit further including a catalyst.

16. The method for closing a wound according to claim 15, wherein said catalyst being a Polyethylene glycol derivative.

17. The method for closing a wound according to claim 9, wherein said method further including the step of:
applying a second transparent support layer to said secured wound prior to applying said flowable adhesive to exposed portions of said support layer and said wound.
